(19) United States
(12) Patent Application Publication
(10) Pub. No.: US 2025/0169951 A1
(43) Pub. Date: May 29, 2025

(54) ADJUSTABLE MITRAL VALVE ANNULOPLASTY DEVICES AND METHODS FOR USE

(71) Applicant: The Board of Trustees of the Leland Stanford Junior University, Stanford, CA (US)

(72) Inventors: Matthew Park, Piedmont, CA (US); Yuanjia Zhu, Stanford, CA (US); Annabel M. Imbrie-Moore, Stanford, CA (US); Y. Joseph Woo, Stanford, CA (US)

(21) Appl. No.: 19/036,936

(22) Filed: Jan. 24, 2025

Related U.S. Application Data
(63) Continuation of application No. PCT/US2023/028899, filed on Jul. 27, 2023.
(60) Provisional application No. 63/392,827, filed on Jul. 27, 2022.

(51) Int. Cl. A61F 2/24

(52) U.S. Cl. CPC A61F 2/2448; A61F 2230/0034; A61F 2250/0004; A61F 2250/0029

(57) ABSTRACT

Adjustable mitral annuloplasty rings are disclosed that include a body including an anterior segment with a hub and first and second curved lateral segments extending from opposite ends of the anterior segment. In one example, the body terminates at tips of the lateral segments generally opposite the anterior segment to define a “C” shape. Alternatively, the body includes a posterior segment extending between ends of the lateral segments opposite the anterior segment to define a “D” shape. One or more channels are provided around a perimeter of the body, e.g., through the segments and communicating with openings in the hub, and a filament is received in the channels such that the filament extends around the perimeter of the body and ends of the filament exit the openings in the hub such that the ends may be tensioned to cause constriction of one or more of the segments of the body.

### RELATED APPLICATION DATA

The present application is a continuation of co-pending International Application no. PCT/US2023/028899, filed Jul. 27, 2023, which claims benefit of U.S. provisional application Ser. No. 63/392,827, filed Jul. 27, 2022, the entire disclosures of which are expressly incorporated by reference herein.

### STATEMENT REGARDING FEDERALLY SPONSORED RESEARCH AND DEVELOPMENT

None.

### TECHNICAL FIELD

The present application relates to medical devices and, more particularly, to annuloplasty devices and to methods for making and using such devices.

### BACKGROUND

Mitral annuloplasty ring implantation is one of the most common mitral valve surgeries to reduce mitral regurgitation by establishing proper leaflet coaptation. Mitral ring sizing is a difficult process whereby a surgeon decides how much to reduce the size of the annulus while the heart is arrested. Methods currently do not exist to adjust the ring size once the heart has been restarted, and small sizing errors often result in persistent regurgitation, which requires the surgeon to rearrest the heart and re-perform the annuloplasty procedure exposing the patient to additional risk.

Therefore, improved devices for mitral valve repair would be useful.

### SUMMARY

The present application is directed to medical devices and, more particularly, to annuloplasty devices and to methods for making and using such devices.

For example, annuloplasty devices are provided that may be implanted within a mitral valve annulus that allow for asymmetric annulus constriction off-bypass. The devices may include a passive tightening mechanism, e.g., that may exit the left atrium of the patient's heart after implantation, allowing for tightening post-implantation, e.g., once the patient is off cardiopulmonary-bypass. The devices may also allow for asymmetric adjustment of the annulus. To accomplish this, the devices may include custom folding and/or semi-flexible material technologies.

Generally, the devices herein include a body including an anterior segment with a hub and first and second curved lateral segments extending from opposite ends of the anterior segment. In one example, the body terminates at tips of the lateral segments generally opposite the anterior segment such that the body defines a generally incomplete ring or “C” shape. In another example, the body includes a posterior segment extending between ends of the lateral segments opposite the anterior segment such that the body is an enclosed ring defining a generally complete ring or “D” shape. One or more channels are provided around a perimeter of the body, e.g., through the anterior, lateral, and/or posterior segments, communicating with openings in the hub, and one or more filaments are received in the one or more channels such that the filament(s) extend around the perimeter of the body and ends of the filament(s) exit the openings in the hub such that the ends may be tensioned to cause constriction of one or more of the segments of the body.

In accordance with one example, an adjustable mitral valve annuloplasty device is provided that includes an annular body comprising an elongate curved posterior segment, first and second curved lateral segments extending from opposite ends of the posterior segment, and an anterior segment extending between ends of the lateral segments opposite the posterior segment; a hub on the anterior segment; one or more channels around a perimeter of the body through one or more of the anterior, lateral, and posterior segments; and one or more filaments received in the one or more channels such that the filament(s) extend around the perimeter of the body and ends of the filament(s) exit the openings in the hub such that the ends may be tensioned to cause constriction of one or more of the segments of the body.

In accordance with another example, an adjustable mitral valve annuloplasty device is provided that includes a body comprising an anterior segment and first and second curved lateral segments extending from opposite ends of the anterior segment; a hub on the anterior segment; one or more channels around a perimeter of the body through the lateral segments; and one or more filaments received in the one or more channels such that the one or more filaments extend at least partially around a perimeter of the body, wherein ends of the one or more filaments exit the openings in the hub such that the ends may be tensioned to cause constriction of one or more of the segments of the body.

In accordance with still another example, a method for performing annuloplasty is provided that includes providing an annuloplasty device comprising a body comprising an anterior segment and first and second curved lateral segments extending from opposite ends of the anterior segment, one or more channels around a perimeter of the body through the lateral segments, and one or more filaments received in the one or more channels such that the one or more filaments extend at least partially around a perimeter of the body and ends of the one or more filaments exit openings in a hub on the anterior segment; positioning the device around a mitral valve annulus such that the anterior segment is located adjacent an anterior leaflet of the valve; and securing the device to tissue adjacent the annulus.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIGS. 1A-1C show an example of an adjustable annuloplasty device including an annular body including a generally “D” shape and a hub for guiding a filament received within the body to allow selective constriction of the device.

FIGS. 2A and 2B show the annuloplasty device of FIGS. 1A-1C implanted around a mitral valve annulus.

FIGS. 3A and 3B show another example of an adjustable annuloplasty device including an annular body including a generally “C” shape and a hub for guiding a filament received within the body to allow selective constriction of the device.

FIGS. 4A-4D shown an exemplary method for implanting the annuloplasty device of FIGS. 3A and 3B around a mitral valve annulus.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

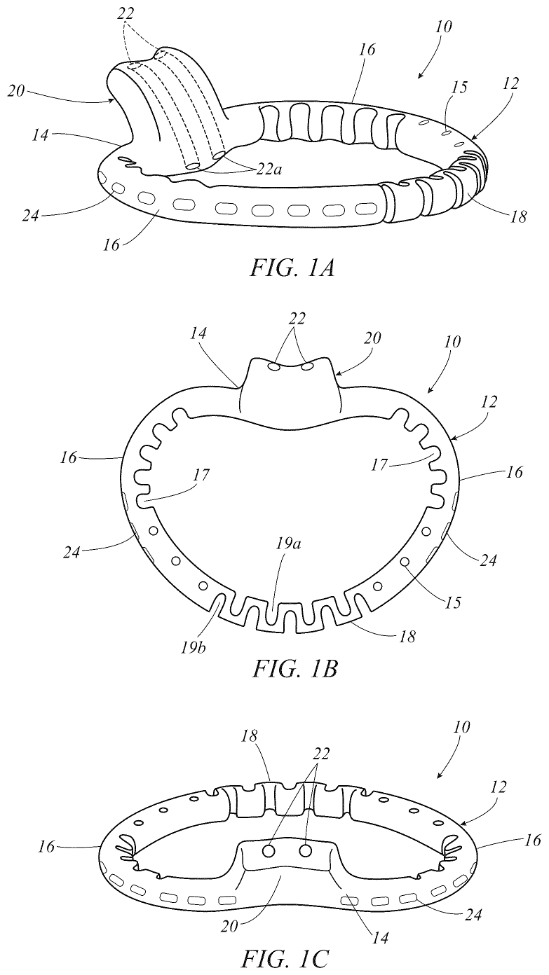

Turning to the drawings, FIGS. 1A-1C show an example of an adjustable mitral valve annuloplasty device 10 including a body 12 that includes an anterior segment 14, first and second curved lateral segments 16 extending from opposite ends of the anterior segment 14, and a curved posterior segment 18 extending between ends of the lateral segments 16 opposite the anterior segment 14, e.g., such that body 12 defines an enclosed ring. In one example, as shown, the segments 14-18 may be shaped such that the body 12 defines a generally “D” shape, e.g., with the posterior segment 18 having a radius of curvature larger than the lateral segments, e.g., as best seen in FIG. 1B. The anterior segment 14 may be substantially straight or may include one or more relatively small curves, i.e., having a radius of curvature larger than the posterior segment 18. For example, as best seen in FIG. 1B, the anterior segment 14 may define a concave outer edge (relative to the enclosed center of the body 12) between the lateral segments 16.

In the example shown, the segments 14-18 may lie generally within a common plane, e.g., as best seen in FIGS. 1A and 1C. Alternatively, one or more of the segments may extend out of the common plane, if desired.

A hub 20 is provided on the anterior segment 14 including a pair of openings 22 therein, and one or more channels 24 are provided around a perimeter of the body 12, e.g., through one or more of the anterior, lateral, and posterior segments 14-18. For example, the channels may include a plurality of lateral perforations 24 spaced apart from one another around a perimeter of the body 12, e.g., in at least the lateral segments 16 and, optionally, in at least a section of the anterior and/or posterior segments 14, 18.

The openings 22 in the hub 20 may communicate with one or more channels in the anterior segment 14 or may simply extend through the hub 20 to opposite openings, e.g., at a lower end of the hub 20. For example, as shown in FIG. 1A, the openings 22 may extend from the upper end of the hub 20 to opposite openings 22a on the lower end of the hub 20, e.g., such that two passages extend through the hub 20 between the upper and lower ends. Alternatively, each opening 22 may communicate with an internal passage in the hub 20 that turns to extend circumferentially towards the respective lateral segments 16.

Alternatively, at least some of the openings 22 may define a discontinuous lumen extending circumferentially around one or more of the segments, e.g., with the openings 22 defining enclosed holes aligned with one another around the circumference of the body 12. In another alternative, at least some of the openings 22 may include recesses extending circumferentially around an outer surface (relative to the enclosed center of the body 12) of one or more of the segments.

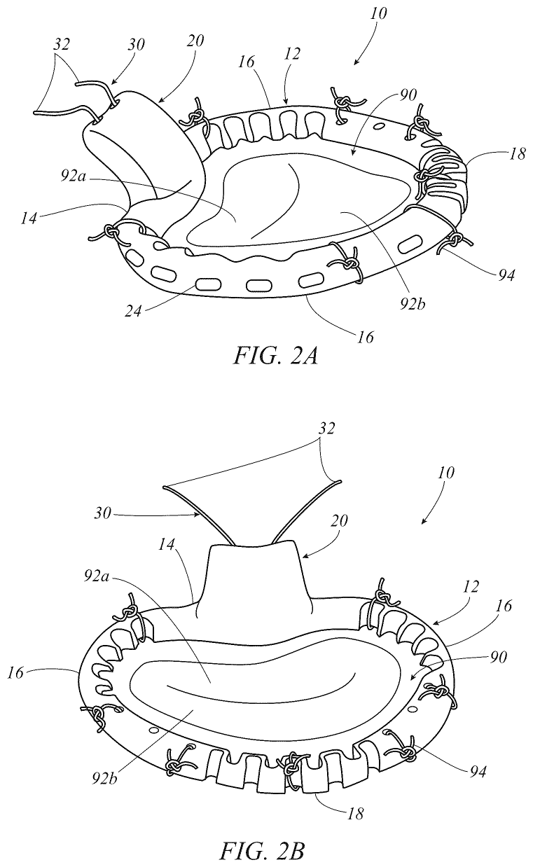

With additional reference to FIGS. 2A and 2B, one or more filaments 30 may be received in the channel(s) 24 such an intermediate region of the filament(s) 30 extends around the perimeter of the body 12 and ends 32 of the filament(s) 24 exit the openings 22 in the hub 20 such that the ends 26 may be tensioned to cause constriction of one or more of the segments of the body 12, as described further elsewhere herein.

For example, if the segments 14-18 of the body 12 lie generally within a plane, the hub 20 may extend from the anterior segment 14 out of the plane, e.g., such that the openings 22 are located above the plane. In this example, the openings 22 may extend transversely relative to the plane through the hub 20 to lower openings 22a. Alternatively, the openings 22 may communicate with channels that extend laterally through the hub 20, e.g., with one channel extending towards each lateral segment (not shown).

As shown in FIGS. 2A and 2B, the filament 30 may include a suture that is threaded through the channels 24 around the perimeter of the body 12. For example, a first end 32 of the suture 30 may extend from a first opening of the openings 22 in the hub 20 and be threaded through the channels 24 around the perimeter of the body 12 such that a second end 32 of the suture 30 extends from a second opening of the openings 22 in the hub 20. Thus, one or both ends 32 of the filament 30 may subsequently be pulled or otherwise tensioned to constrict the body 12.

Optionally, one or more holes or passages 15 may be provided through one or more of the segments for receiving a suture or other fastener (not shown). For example, as shown, each of the lateral segments 14 include a plurality of holes 15, e.g., extending from the upper surface through to the lower surface, which may facilitate introducing a suture through the segments 14, as described elsewhere herein.

To facilitate constriction of the body 12, the lateral and/or posterior segments 14, 18 may include a plurality of geometric features such as grooves, kerfing, and/or other features therein that allow or facilitate the radial length and/or radius of curvature of the segments to be reduced, e.g., by pulling the ends 32 of the filament 30. For example, as best seen in FIG. 1B, each of the lateral segments 16 may include a plurality of grooves 17 formed therein, e.g., a plurality of grooves 17 that are spaced apart from one another along an inner surface (relative to the enclosed center of the body 12) of each of the lateral segments 16. The grooves 17 may extend only partially through the width of the lateral segments 14 from the inner surface partially towards the outer surface to define a plurality of hinges that facilitate bending of the lateral segments 14, e.g., to reduce their radius of curvature. Thus, the channels 24 may be provided within the outer surface while the grooves 17 may be provided on the inner surface without interfering with one another.

In addition or alternatively, the posterior segment 18 may include a plurality of grooves 19 formed therein, e.g., to accommodate radial constriction of the posterior segment when the filament 30 is tensioned. For example, as shown the posterior segment 18 may include a plurality of inner grooves 19a spaced apart from one another along an inner surface of the posterior segment 18, and a plurality of outer grooves 19b spaced apart from one another along an outer surface of the posterior segment 18, e.g., with the inner and outer grooves 19a, 19b alternating along a section of the posterior segment 18. The grooved section of the posterior segment 18 may not include any channels for the filament, e.g., such that the filament extends around the perimeter of the body 12 outside this section, e.g., as shown in FIG. 2B. Alternatively, one or circumferential channels may be provided through this section, e.g., aligned with one another around the length of the posterior segment 18, to receive the filament (not shown).

The body 12 and hub 20 may be integrally formed together from a single piece of material or separate portions that are attached together. In one example, the segments 14-18 of the body 12 and the hub 20 may be integrally formed together, e.g., from the same material, for example, by one or more of molding, casting, forming, 3D-printing, machining, and the like. For example, the body 12 may be molded or 3D-printed to include all of the features, e.g., the channels 24 and/or grooves 17, 19. Alternatively, the body 12 may be formed without the channels 24 and/or grooves 17, 19, which may subsequently be formed in the body material, e.g., by machining, laser cutting, and the like. In a further alternative, at least the anterior segment 14 may be formed separately from the lateral segments 16, which may be permanently attached together, e.g., by one or more of cooperating connectors, interference fit, bonding with adhesive, fusing, welding, and the like. The device 10 may be formed from biocompatible materials, such as plastic material, e.g., silicone, urethane, polyurethane, polyethylene, and the like, metal material, e.g., Nitinol, Elgiloy, and the like, or composite materials.

In one example, the device may be formed from plastic material, e.g., having a Shore hardness ranging from 10A to 40A. Optionally, the anterior segment 14 may be formed from material that has greater rigidity than the lateral and posterior segments 16, 18. For example, the anterior segment 14 may be formed from separately from substantially rigid material, and then lateral and posterior segments 16, 18 may be formed together from semi-rigid or flexible material, and the lateral segments 16 may be permanently attached to the anterior segment 14, e.g., on opposite sides of the hub 20. In this example, any tension applied to the filament(s) 30 may translate through the anterior segment 14 to the anterior and posterior segments 16, 18, e.g., to facilitate constriction and/or other deflection of those segments.

The outer surface of the body 12 may be covered with fabric, e.g., Dacron and the like, e.g., to receive fasteners during implantation and/or to promote tissue ingrowth after implantation. Optionally, a sewing cuff (not shown) of material that may be punctured with a suture (i.e., fabric or a polymer) may be incorporated into the device 10 (or any of the other devices herein) and/or attached around all or a portion of a perimeter of the device 10 to facilitate the repair procedure. For example, a cuff may be provided that at least partially surrounds the device or resides on the inner, outer, top, and/or bottom regions of device. Optionally, the sewing cuff or outer fabric may be marked with colors (not shown), e.g., to indicate useful landmarks on the device 10, e.g., the location of the outer edges of the lateral segments 16, the center of the posterior segment 18, and the like.

Thus, the assembled device 10 may include a body 12 covered with fabric with the ends of the filament(s) 32 extending from the hub 20 (through any fabric covering the hub 20). The lengths of the ends of the filament(s) 32 may have sufficient length to extend from a patient's heart and/or body when the body 12 is positioned adjacent a valve annulus being repaired. Optionally, a single size device may be provided or, alternatively, several different size devices (not shown) may be provided such that a surgeon may select a desired size device to accommodate an individual patient's anatomy.

With particular reference to FIGS. 2A and 2B, the annuloplasty device 10 may be implanted around a mitral valve annulus 90, e.g., to reduce mitral valve regurgitation and/or otherwise repair the valve. Initially, the mitral valve may be accessed using conventional open or minimally invasive procedures, e.g., with the patient's heart arrested using conventional by-pass systems and methods.

Once the valve is exposed, the device 10 may be positioned around the valve annulus 90 such that the anterior and posterior segment 14, 18 are positioned opposite one another, e.g., above or otherwise adjacent the anterior and posterior leaflets 92a, 92b, respectively, of the valve. The device 10 may then be secured to tissue adjacent the annulus 90 using one or more fasteners. For example, as shown, suturing may be used, e.g., by introducing sutures 94 through fabric covering the device 10 and/or through one or more passages in the body 12 into the adjacent tissue. Optionally, one or more holes or passages may be provided through the body 12 to facilitate introducing a suture or other fastener therethrough to secure the device 10. For example, as shown, a suture 94 may be introduced through the overlying fabric through a hole (not shown, corresponding to the hole 15 shown in FIGS. 1A and 1B) to facilitate securing the device 10 to the valve annulus 90. In addition or alternatively, sutures may be secured around or through other locations on the body 12, e.g., corresponding to the grooves 17 in the lateral segments 14.

During implantation, the ends 32 of the suture 30 may extend from the annulus 90, e.g., from the left atrium of the heart and out of the patient's body (not shown). At any time during implantation, if desired, the ends 32 of the suture 30 may be tensioned to constrict the body 12, e.g., before, during, or after suturing the device 10 around the annulus 90. For example, after securing the device 10 around the annulus 90, the ends 32 may be pulled to cause substantially uniform constriction of the body 12, e.g., through the lateral and posterior segments 16, 18, and thereby constrict or otherwise support the valve annulus 90. Alternatively, one of the ends 32 may be pulled to cause preferential constriction of the corresponding lateral segment 16, e.g., to provide asymmetrical constriction of the body 12, if desired.

Further, once implantation is complete and the patient is removed from cardiopulmonary bypass, the device 10 may be adjusted further, if desired. For example, once bypass is discontinued and/or the heart begins beating, the mitral valve may be monitored to confirm whether the repair has been successful. If regurgitation persists, one or both of the ends 32 may be tensioned further to constrict one or more of the segments of the body 12 until the valve performs properly. The ends 32 of the suture 30 may then be secured relative to the hub 20, e.g., by advancing one or more knots (not shown) down each end to secured the knot(s) against the respective openings 22 in the hub 20. Alternatively, the ends 32 may be knotted or otherwise tied together and secured to the hub 20. In addition or alternatively, one or more fasteners (not shown) may be used to secure the ends to the hub 20 and/or fabric covering. Any excess filament from the ends 32 may be then be cut or otherwise severed and removed. The procedure may then be completed using conventional methods.

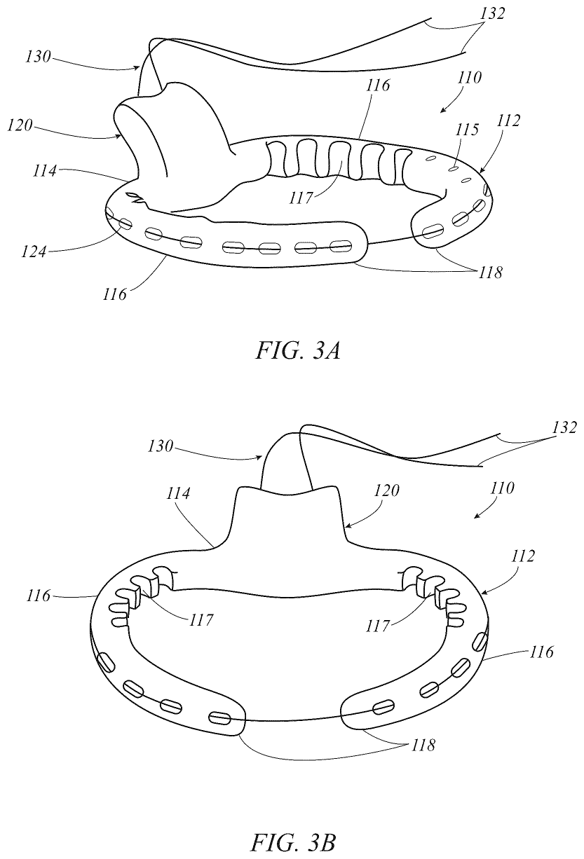

Turning to FIGS. 3A and 3B, another example of an adjustable mitral valve annuloplasty device 110 is shown that includes a body 112 that includes an anterior segment 114 and first and second curved lateral segments 116 extending from opposite ends of the anterior segment 14, which may include a plurality of grooves 117, e.g., spaced apart along the inner surface of the lateral segments 116, similar the grooves 117 in the device 10. Unlike the device 10, the lateral segments 116 terminate at tips 118. In a relaxed state, e.g., as shown, the tips 118 may be spaced apart from one another such that the body 112 defines a generally “C” shape, e.g., lying generally within a common plane.

Similar to the device 10, a hub 120 is provided on the anterior segment 114 including a pair of openings 122 therein, and one or more channels 124 are provided around a perimeter of the body 112, e.g., through the anterior and/or lateral segments 114, 116. For example, the channels may include a plurality of lateral perforations 124 spaced apart from one another circumferentially around an outer surface of the lateral segments 116. One or more filaments, e.g., suture 130, may be received in the channels 124, e.g., threaded in and out of the channels 124 (or otherwise through enclosed lumens or other channels) around the lateral segments 116, also similar to the device 10.

In the example shown, a single filament 130 may be used with opposite ends 132 of the filament 130 extending from respective openings 122 in the hub 120, and the filament 130 threaded through the channels 124 around the perimeter of the body 112, e.g., such that an intermediate region of the filament 130 extends between the tips 118 of the lateral segments 116, as shown. Alternatively, two filaments may be used, e.g., with a first filament extending from a first opening of the openings 122 through the one or more channels in the first lateral segment 116 and a second filament extending from a second opening of the openings 122 through the one or more channels in the second lateral segment 116 (not shown). In this alternative, ends of the filaments may be secured adjacent the tips 118 of the respective lateral segments 116, e.g., such that each lateral segment 116 may be constricted independent of the other, if desired. Alternatively, if a single filament 130 is provided that extends between the tips 118 of the lateral segments 116, one or more knots may be provided (not shown) that may contact the tips 118, e.g., to transfer tension applied to only one end 132 of the filament 130 to be limited only to the respective lateral segment 116, if desired.

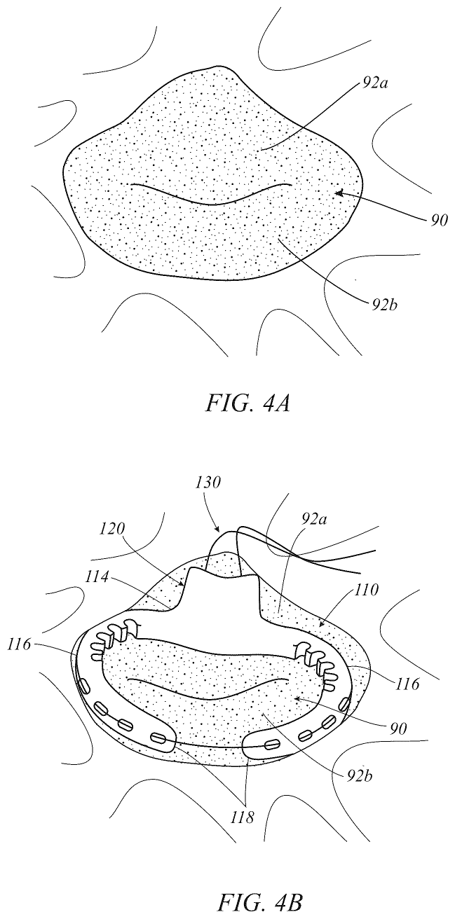

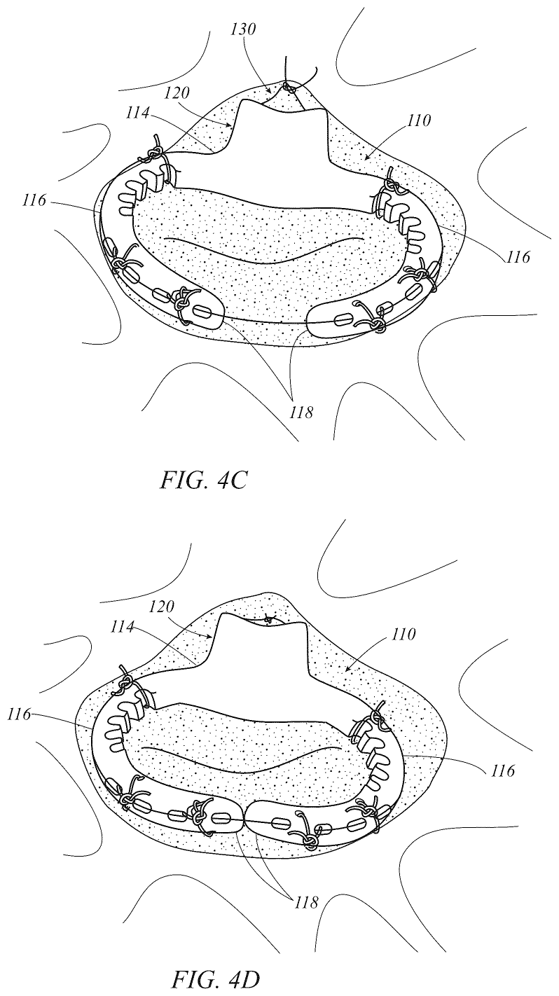

Turning to FIGS. 4A-4D, an exemplary method is shown for implanting the annuloplasty device 110 around a mitral valve annulus 90, e.g., to reduce mitral valve regurgitation and/or otherwise repair the valve. Initially, as shown in FIG. 4A, the mitral valve may be accessed using conventional open or minimally invasive procedures, e.g., with the patient's heart arrested using conventional by-pass systems and methods.

Once the valve is exposed, as shown in FIG. 4B, the device 110 (shown with the fabric covering omitted) may be positioned around the valve annulus 90 such that the anterior segment 114 is positioned adjacent the base of the anterior leaflet 92a of the valve, and the tips 118 of the lateral segments 116 are positioned adjacent the posterior leaflet 92b of the valve. The device 110 may then be secured to tissue adjacent the annulus 90 using one or more sutures and/or other fasteners 94. For example, one or more sutures may be introduced through fabric covering the device 110 and/or through one or more passages in the body 112 into the adjacent tissue (not shown).

During implantation, as shown in FIG. 4B, the ends 132 of the filament 130 may extend from the annulus 90, e.g., from the left atrium of the heart and/or out of the patient's body (not shown). At any time during implantation, the ends 132 of the filament 130 may be tensioned to constrict the body 112, e.g., before, during, or after suturing the device 110 around the annulus 90. For example, as shown in FIG. 4C, initially the tips 118 of the lateral segments 116 may be spaced apart from one another when the device 110 is sutured around the annulus 90. The ends of the filament 130 may be tensioned to constrict the device 110 substantially uniformly, thereby pulling the tips 118 closer to one another, e.g., until they abut one another as shown in FIG. 4D, or otherwise to constrict or otherwise manipulate the valve annulus 90. Alternatively, as described previously, tension may be applied to only one of the ends 132, desired, e.g., to constrict and/or otherwise deflect the respective lateral segment 116 independently of the other lateral segment 116, e.g., if asymmetrical constriction is desired.

Further, once implantation is complete and the patient is removed from bypass, the device 110 may be adjusted further, similar to the device 10. For example, once bypass is discontinued and/or the heart begins beating, the mitral valve may be monitored to confirm whether the repair has been successful. If regurgitation persists, one or both of the ends 132 may be tensioned further to constrict one or more of the segments of the body 112 until the valve performs properly. The ends 132 of the suture 130 may then be secured relative to the hub 20, e.g., tied in a knot 133 as shown in FIG. 4A, and any excess filament from the ends 132 may be then be cut or otherwise severed and removed. The procedure may then be completed using conventional methods.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. An adjustable mitral valve annuloplasty device, comprising:
an annular body comprising an elongate curved posterior segment, first and second curved lateral segments extending from opposite ends of the posterior segment, and an anterior segment extending between ends of the lateral segments opposite the posterior segment;
a hub on the anterior segment;
one or more channels around a perimeter of the body through one or more of the anterior, lateral, and posterior segments; and
a filament received in the one or more channels such that the filament extends around the perimeter of the body and ends of the filament exit the openings in the hub such that the ends may be tensioned to cause constriction of one or more of the segments of the body.

2. The device of claim 1, wherein the body defines a generally “D” shape within a plane.

3. The device of claim 2, wherein the hub extends from the anterior segment out of the plane such that the openings are located above the plane.

4. The device of claim 1, wherein each of the lateral segments include a plurality of grooves formed therein to accommodate radial constriction of the lateral segments when the filament is tensioned.

5. The device of claim 4, wherein the grooves are spaced apart from one another along an inner surface of each of the lateral segments.

6. The device of claim 1, wherein the posterior segment includes a plurality of grooves formed therein to accommodate radial constriction of the posterior segment when the filament is tensioned.

7. The device of claim 6, wherein the posterior segment grooves comprise a plurality of inner grooves spaced apart from one another along an inner surface of the posterior segment.

8. The device of claim 7, wherein the posterior segment grooves further comprise a plurality of outer grooves spaced apart from one another along an outer surface of the posterior segment.

9. The device of claim 8, wherein the inner and outer grooves alternate along a section of the posterior segment.

10. The device of claim 1, wherein the one or more channels comprise a plurality of lateral perforations spaced apart from one another around the perimeter in the lateral segments to slidably receive the filament therein.

11. The device of claim 1, further comprising one or more holes through the body to facilitate introducing a fastener through the body.

12. The device of claim 11, wherein the one or more holes comprise a plurality of holes spaced apart from one another in the lateral segments.

13. The device of claim 12, wherein the plurality of holes extend from an upper surface of the lateral segments through to a lower surface of the lateral segments.

14. The device of claim 1, wherein the anterior segment has a greater rigidity than the lateral and posterior segments.

15. The device of claim 1, wherein the filament is secured to one or more of the lateral and posterior segments such that one end of the filament may be tensioned to apply a compressive force to the respective lateral segment.

16. The device of claim 1, wherein the filament is threaded in and out of the channels around each of the lateral segments.

17. The device of claim 1, wherein the filament extends along the posterior segment between the lateral segments outside the posterior segment.

18. The device of claim 1, wherein the filament extends along the posterior segment between the lateral segments through one or more circumferential passages in the posterior segment.

19-20. (canceled)

21. An adjustable mitral valve annuloplasty device, comprising:
a body comprising an anterior segment and first and second curved lateral segments extending from opposite ends of the anterior segment;
a hub on the anterior segment;
one or more channels around a perimeter of the body through the lateral segments; and
one or more filaments received in the one or more channels such that the one or more filaments extend at least partially around a perimeter of the body, wherein ends of the one or more filaments exit the openings in the hub such that the ends may be tensioned to cause constriction of one or more of the segments of the body.

22-46. (canceled)

47. A method for performing annuloplasty, comprising:
providing an annuloplasty device comprising a body comprising an anterior segment and first and second curved lateral segments extending from opposite ends of the anterior segment, one or more channels around a perimeter of the body through the lateral segments, and one or more filaments received in the one or more channels such that the one or more filaments extend at least partially around a perimeter of the body and ends of the one or more filaments exit openings in a hub on the anterior segment;
positioning the device around a valve annulus such that the anterior segment is located adjacent an anterior leaflet of the valve; and
securing the device to tissue adjacent the annulus.

48-59. (canceled)
